(19) United States
(12) Patent Application Publication
(10) Pub. No.: US 2010/0197906 A1
(43) Pub. Date: Aug. 5, 2010

(54) Method for purification of chlorinated sucrose derivatives from reaction mixture by chromatography

(75) Inventors: Subramaniyam, Mumbai (IN); Chethana, Mumbai (IN); Rakesh Ratnam, Mumbai (IN); Sundeep Aurora, Mumbai (IN)

Correspondence Address: THE NATH LAW GROUP, 112 South West Street, Alexandria, VA, 22314, US

(73) Assignee: PHARMED MEDICARE PRIVATE LIMITED, Mumbai, Maharashtra (IN)

(21) Appl. No.: 11/921,564

(22) PCT Filed: Jun. 2, 2006
(86) PCT No.: PCT/IN2006/000187

§ 371 Date: Dec. 5, 2007

(30) Foreign Application Priority Data
Jun. 6, 2005 (IN) 673/MUM/2005

(51) Int. Cl. B01D 15/08; C07H 5/02

(52) U.S. Cl. 536/122; 210/656

(57) ABSTRACT

A chromatographic process of DMF removal from an aqueous composition is described comprising its loading on a column of hydrophobic fixed bed adsorbent and eluting out DMF with an aqueous alkaline buffer. This method is useful to remove DMF as a process of general application wherever simultaneous removal and isolation of DMF is desired from an organic molecule which is not an organic solvent, is soluble in DMF. This method can be used for simultaneous removal of DMF from reaction mixtures and isolation of Trichlorogalactose (TGS) or TGS-6-acetate in a process of production of TGS.

### TECHNICAL FIELD

The present invention relates to a process and a novel strategy for isolation and purification of 1′-6′-Dichloro-1′-6′-DIDEOXY-β-Fructofuranasyl-4-chloro-4-deoxy-galactopyranoside (TGS) and other chlorinated sucrose derivatives from a reaction mixture.

1. Background of Invention

Chlorinated sucrose preparation is a challenging process due to the need of chlorination in selective less reactive positions in sucrose molecule in competition with more reactive positions. Generally, this objective is achieved by a procedure which involves essentially protecting the hydroxy group in the pyranose ring of sugar molecule by using various protecting agents alky/aryl anhydride, acid chlorides, orthoesters etc., and the protected sucrose is then chlorinated in the desired positions (1′-6′ &, 4) to give the acetyl derivative of the product, which is then deacylated to give the desired product 1′-6-Dichloro-1-6-DIDEOXY-β-Fructofuranasyl-4-chloro-4-deoxy-galactopyranoside i.e. 4,1′,6′ trichlorogalactosucrose (TGS).

Strategies of prior art methods of production of TGS are based on following: Sucrose-6-acetate is chlorinated by Vilsmeier-Haack reagent to form TGS-6-acetate. After chlorination, the deacetylation of TGS-6-acetate to TGS is carried out in the reaction mixture itself. The process is described in greater details in several patents and patent applications including Ratnam et al (2005) in WO2005090374 and Ratnam et al (2005) in WO2005090376. As an inevitable part of above synthesis strategy, various other chloro substitution products are also produced in varying amounts. Isolation of TGS from the other substitution impurities by liquid-liquid extraction is usually a daunting task due to affinity of the product to hydrophilic as well as hydrophobic solvents. Removal of N,N-dimethylformamide (DMF) in such reaction mixtures is also a strategically important job which is usually done by adopting several methods. DMF removal becomes strategically important because its traces interfere with isolation of chlorinated sucrose or their derivatives from the reaction mixture by the known methods of separation such as solvent extraction and crystallization. Removal of DMF is an equally difficult task because it is a high boiling solvent as well as has partial solubility in aqueous as well as organic solvents. High boiling nature rules out any distillation at high temperature as it shall lead to charring of a chlorinated sucrose or its precursor/ester. To complicate the situation, TGS has solubility in DMF as well as water. DMF has been removed by Navia et al (1996) in U.S. Pat. No. 5,498,709 and by Navia et al (1996b) in U.S. Pat. No. 5,530,106 by steam distillation. However, this method leads to several fold increase in the volume of the reactants due to condensation of the steam. In this context, application of column chromatography on silanized silica gel or an appropriate resin followed by elution by an alkaline buffer is found in this invention to be a far more efficient and simpler option than other options in achieving removal of DMF as well as isolation of chlorinated sucrose simultaneously directly from chlorination reaction mixture, before as well as after de-esterification, with as well as without prior removal of DMF by one or the other process of its removal.

2. Prior Art

TGS was isolated by Mufti et at (1983) in U.S. Pat. No. 4,380,476 without undue difficulty from deacylated mixture of chlorinated sucrose derivatives obtained by chromatography on silica gel by using chloroform: acetone mixture as eluent, a 2:1 mixture followed by 1:1 mixture, TGS is eluted in 1:1 mixture. Khan et al (1992) in U.S. Pat. No. 5,136,031; by Dordick et al (1992) in U.S. Pat. No. 5,128,248, Walkup et al (1990) in U.S. Pat. No. 4,980,463, Jenner et al (1982) in U.S. Pat. No. 4,362,869 and Catani et al (1999) in U.S. Pat. No. 5,977,349 used the same method for isolating TGS from their reaction mixture.

Mufti et al (1982) also reported use of Dowex 50×4, 50 to 100 mesh (dry), a polystyrene sulphonic acid cation exchange resin cross linked with divinyl benzene for above purpose. Catani et al. (1999) in U.S. Pat. No. 5,977,349 also disclosed use of porous gel cation exchange resins as adsorbent, particularly polystyrene-based sodium sulphonic resin crosslinked with 4% divinylbenzene as adsorbent and water as desorbent.

These were however, cumbersome, inconvenient and expensive in actual practice. A need for chromatographic methods with more convenience, better efficiency combined with relatively low cost of operation was felt.

### SUMMARY OF INVENTION

It was found that improvement in efficiency and lowering of cost of separation of DMF from liquid compositions containing DMF and one or more of a component comprising chlorinated sucrose or their precursors/derivatives including esters, impurities including salts were achieved by column chromatography when hydrophobic adsorbents, which included silanated silica gel, a non-ionic polyacrilyc based resin ADS600 and the like, were used as fixed bed adsorbents and alkaline buffers over a wide range of pH, including 9.5 to 11.5, were used as eluents. Water, preferably demineralized water, also gives separation on above mentioned adsorbents to enable removal of DMF, although alkaline buffer gives better results as eluent.

### DETAILS OF THE INVENTION

Throughout this specification and claims, a mention in singular is also deemed to cover its plural, unless context suggests otherwise. Thus, mention of “a method of isolation and purification” also includes more than one and all methods of isolation and purification.

Further, the examples, techniques used, chemicals used are meant to illustrate how the invention works and they do not constitute limitations of the invention. Any modification, adaptation or equivalent of the same obvious to an average person skilled in the art is also included within the scope of this invention if the same is covered by the scope of the claims.

The strategy of various processes for production of TGS based on chlorination of sucrose-6-ester, as described earlier, invariably included removal of DMF at the end or before deacylation by a suitable method and then the reaction mixture was taken further for purification and isolation of TGS-6-acetate or TGS by one or more of a method of isolation and purification.

In tune with that, it was found that a TGS-6-acetate or TGS could be very efficiently separated by adsorption on a fixed bed adsorbent, which could be subsequently eluted out by alkaline buffers when a reaction mixture containing them and from which DMF was removed was loaded on the fixed bed. This invention is subject matter of a co-pending International application no. PCT/IN05/00409 with International filing date of 9 Dec. 2005 and having priority date of 10 Dec. 2004 from an Indian patent application no. 1317/MUM/2004 and the same is expressely incorporated here by way of reference. Silanated silica gel was used as an adsorbent very successfully in this invention.

Removal of DMF was until then considered such an integral and inseparable part of the process of production of TGS that in initial work, only those mixtures were loaded from which substantial portion of DMF was removed by applying one or another method of DMF removal.

It was very soon invented, however, contrary to the established concepts by then, that hydrophobic adsorbents for adsorption of TGS-6-acetate or

TGS and elution of the same by alkaline buffers is equally possible even if DMF was not removed. Thus, the method invented for isolation of TGS-6-acetate and/or TGS could be very efficiently used as a primary method for DMF removal too. Since DMF is a medium for reactions in several organic reactions and its removal could pose as vital a problem as in case of TGS manufacture, this invention is useful even for reactions other than TGS manufacture wherever DMF removal is the primary objective and the reaction mixture may not contain TGS-6-acetate or TGS and may contain any other organic compound. Thus, embodiments of this invention applied to reaction mixtures containing TGS-6-acetate or TGS are also illustrative of a process of DMF removal which can be applied to remove DMF from reaction mixtures having any organic compound other than TGS-6-acetate or TGS having a physical or a chemical property analogous to TGS-6-acetate or TGS. In this perspective, the process of this invention, i.e. use of a hydrophobic adsorbent as a fixed bed adsorbent and alkaline buffer as an eluent is in fact a very effective process of removal of DMF from any liquid mixture containing DMF when other components of the mixture are such that they adsorb or retard substantially on the fixed bed adsorbent used and allow DMF to pass through with the alkaline elution buffer.

Adsorbents useful as fixed bed adsorbents for above purpose include silanated silica gel, a special resin called ADS600 which is polyacrylic based one and the like.

Embodiment of this invention lies in a process involving use of one or more of a hydrophobic adsorbents as a fixed bed adsorbent, loading of the neutralized liquid solution of reactants comprising one or more of components capable for adsorbing on a hydrophobic fixed bed adsorbent including TGS, salts, impurities and DMF on to the fixed bed and capable of getting eluted with an alkaline buffer such as to get most of the DMF washed out of the column with the alkaline eluent earlier than the chemical constituents adsorbed or retarded on the said hydrophobic fixed bed adsorbent and the said adsorbed or retarded chemical constituents adsorbed or retarded on the said hydrophobic fixed bed adsorbent is eluted out in a subsequent fraction which contains no or only a trace of DMF. Thus, use of these adsorbents shall work also as very effective methods for removal of DMF too.

In this work, silanated silica gel was used as a fixed bed hydrophobic adsorbent, the neutralized reaction mass from which DMF was removed substantially was loaded at about pH 7.5 and and alkaline buffer, preferably of pH 9.5 to 10.5 was used for elution.

DMF removal can also be achieved by several other methods including subjecting the neutralized reaction mass to Agitated Thin Film Drying (Ratnam et al (2005) WO 2005090374) wherein all the liquid reaction mixture is dried under mild and quick drying conditions and solids are obtained. The solids can then be dissolved in water, extracted into a water immiscible solvent such as ethyl acetate, etc and then subjected to concentration till a final syrup containing TGS and a mixture of chlorinated sucrose derivatives are obtained. This syrup was then loaded on to silanized silica gel packed in an appropriate column where the TGS is purified.

The liquid compositions which can be subjected to removal of DMF by column chromatography can also originate as a solution of 6-acetyl-TGS or TGS prepared in water or suitable solvents or as a process stream from a process of production of 6-acetyl-TGS or TGS. The said process of production of TGS-6-acetate or TGS includes a process described by Mufti et al. (1983) U.S. Pat. No. 4,380,476, Walkup et al. (1990) U.S. Pat. No. 4,980,463), Jenner et al. (1982) U.S. Pat. No. 4,362,869, Tulley et al. (1989) U.S. Pat. No. 4,801,700, Rathbone et al. (1989) U.S. Pat. No. 4,826,962, Bornemann et al. (1992) U.S. Pat. No. 5,141,860, Navia et al. (1996) U.S. Pat. No. 5,498,709, Simpson (1989) U.S. Pat. No. 4,889,928, Navia (1990) U.S. Pat. No. 4,950,746, Neiditch et al. (1991) U.S. Pat. No. 5,023,329, Walkup et al. (1992) 5,089,608, Dordick et al. (1992) U.S. Pat. No. 5,128,248, Khan et al. (1995) U.S. Pat. No. 5,440,026, Palmer et al. (1995) U.S. Pat. No. 5,445,951, Sankey et al. (1995) U.S. Pat. No. 5,449,772, Sankey et al. (1995) U.S. Pat. No. 5,470,969, Navia et al. (1996) U.S. Pat. No. 5,498,709, Navia et al. (1996) U.S. Pat. No. 5,530,106 and patent applications containing similar patentable matter including in co-pending application Nos. WO 2005/090374 A1 and WO 2005/090376 A1.

In the following description are given some examples to illustrate basic working of this invention. The reactants used, proportions of reactants used and conditions of chromatography given in the example are only illustrative and are not to be construed to limit the scope of this specification in any way. Any reasonable modifications in the methods described which is obvious to a person skilled in the art, are of analogous and generic in nature, are construed to be within the scope of this invention.

The said liquid compositions could be applied to column either directly without any further processing or modification in them, or after one or more of a process step applied to them to modify their nature including concentration, partial removal of DMF b_{y} distillation under reduced pressure, by solvent extraction, molecular separation and the like.

In one embodiment of invention embodied in this specification, the neutralized reaction mass after chlorination is directly loaded on to the silanized silica gel packed in a column wherein the DMF is also separated and the TGS is isolated simultaneously. This process bypasses the DMF removal step through a process such as Agitated Thin Film Drying for the removal of DMF and also any extraction step prior to purification of TGS.

During the process of chromatographic separation, the mobile phase used was an alkaline buffer at pH ranging between 9 to 12.0 more preferably between 10.5 to 11.5.

The appropriate resin for such separation could be a hydrophobic resin such as polystyrene based resins. The resin on which we have carried out trials is obtained from Thermax named as ADS600.

It is finding of this invention that ADS600, which is a polyacrylic based resin, is useful for the separation/removal/recovery of DMF from other organic molecules, other than an organic solvent, including chlorinated sucrose and their derivatives.

This method can also be adaptively applied and extended to application to separation, isolation and purification of reaction mixtures after one or more purification and modifying steps including DMF removal by other methods.

Examples given in this specification illustrate working of this invention without limiting in any way the actual reactants, techniques, reaction conditions used; any adaptation, variation and equivalent obvious to an average person skilled in the art is covered within the scope of this disclosure/specification within the scope of the claims.

TGS is prepared in most of the processes currently followed amongst many other approaches of its synthesis, by the chlorination of sucrose-6-acetate by the Vilsmeier-Haack reaction. The isolation of TGS from the neutralized mass after chlorination proceeds after the removal of the tertiary amide such as DMF, etc from the neutralized mass

The removal of DMF has been described by Navia et al (1996) in U.S. Pat. No. 5,498,709 by steam distillation. An alternate process of removal of DMF wherein a drier is used to dry the chlorinated reaction mass has been described by Ratnam et al (2005) in WO2005090374 and Ratnam et al (2005) in WO2005090376. The use of Reverse Osmosis has also been described to achieve molecular level separation for the removal of DMF is subject matter of International patent application no. PCT/IN06/00058 having International filing date of 20 Feb. 2006 and priority date of 22 Feb. 2005 derived from Indian patent application no. 198/MUM/2005.

Usually DMF removal is a great problem and holds the key for further purification. This specification embodies an invention wherein the separation of DMF from the reaction mass as well as isolation and purification of the product chlorinated sucrose is achieved by column chromatography using hydrophobic silica gel in a surprisingly simple and more convenient method of achieving DMF removal and further purification process.

The neutralized reaction mass containing the chlorinated sucrose derivatives along with DMF is loaded directly on the hydrophobic silica column. The hydrophobic silica is the stationary phase and the mobile phase used was 100% aqueous buffer at pH ranging from 4-12 more preferably between 8 to 12 and most preferably between 10-11.

In another embodiment, the use of equipments such as Rising Film Evaporators (RFE), Falling Film Evaporators (FFE) or other liquid-liquid extraction systems are used for effective solvent removal at reduced time intervals to avoid exposure of the desired product to higher temperatures.

The method of producing the hydrophobic silica is already described in the already referred International patent application no. PCT/IN05/00409. This hydrophobic silica is packed in a SS column and equilibrated with aqueous buffer solution at pH 10-11 up to 2-3 column volumes. The neutralized mass at pH 7.5 is loaded on the top of the stationary phase. The ratio of the neutralized mass volume to hydrophobic silica ranges from 0.2 to 1.5 times v/w. The loaded neutralized mass is allowed to pass through the silica matrix. After the neutralized mass passes completely into the column, the buffer solution at pH 10-11 is passed through the column continuously. Fractions are collected at the bottom of the column and were analyzed periodically by HPLC and GC. The flow rate was adjusted as per the column dimensions and neutralized mass loading. Those skilled in the art will make the necessary adjustment of parameters.

The initial fractions collected were enriched with DMF. No TGS elution was observed till about 95% of the DMF passed through the column. TGS along with dichloro sucrose derivatives started appearing followed by pure TGS fractions with a DMF content less than 0.02 to 0.2% by wt.

The pure TGS fraction was concentrated by various methods including Reverse Osmosis (RO) system, where the pure TGS was concentrated up to 40% concentration by weight. The residual DMF left over was separated in RO during the concentration of the product fractions. The concentrated product in water was extracted into organic solvent such as ethyl acetate, methyl ethyl ketone, butyl acetate, etc. The solvent extract was charcoalized, concentrated and crystallized.

Hydrophobic silanated silica gel column can be used for DMF separation as well as TGS in following as well as analogous circumstances and all these applications are embodiments of this invention :
a) Neutralized reaction mass can be concentrated up to complete water removal and a mixture of DMF, inorganic salts and chlorinated sucrose derivatives obtained is taken for column chromatography using hydrophobic silica.
b) The neutralized reaction mass can be extracted into a water immiscible or sparingly soluble solvent such as ethyl acetate, methyl ethyl ketone, butyl acetate, etc followed by concentration for the organic solvent removal. The resultant aqueous syrup with DMF and chlorinated sucrose derivatives is purified by column chromatography using hydrophobic silica.
c) The neutralized mass can be concentrated up to complete water removal and then extracted into organic solvent such as ethyl acetate, butyl acetate, methyl ethyl ketone, etc. followed by concentration for the organic solvent removal and syrup obtained is purified by column chromatography using hydrophobic silica

The concentration of the neutralized mass or the concentration of the organic solvent extract in the above said processes has to be carried out under specialized evaporation conditions to avoid any product loss and also effective solvent recovery in the industrial scale. The exposure of TGS to higher temperatures during water or solvent removal is not desirable and hence the use of certain sophisticated distillation systems are used. These are equipments such as the Rising Film Evaporators, Falling film Evaporators, or any such liquid-liquid extraction systems. These equipments enable faster distillation under vacuum and the residence time of the product exposure to the temperature is greatly reduced. This results in enhanced product recovery with minimal loss. Also the efficiency of solvent recovery increases with possible avoidance of decomposition.

Eluent in this invention could be water made alkaline, preferably a buffer in a preferred pH range of 7 to 12, more preferably 9.5 to 11.5, more preferably from 10.5 to 11.5. Eluent may also be water with acetonitrile or acetone preferably in 5% concentration v/v, or methanol in water preferably 2% concentration v/v, or an organic solvent miscible in water in any ratio.

Various methods of silanization of silica gel are reported in United States Pharmacopoea, and (X. S. Zhao and G. Q. Iu,1998, J. Phys. Chem. B 1998, 102,1556-1561). They included the following:

Silanization of silica gel is carried out by allowing the vapors of the silanating agent such as trimethylchlorosilane, dimethyldichlorosilane to coat on to the silica gel in a closed environment. This process takes long hours usually between 6 to 48 hours. After the silanization, the silica is dispersed in water and the Silanized silica gel floats at the top of the solution. This silica is skimmed off and dried before usage in chromatography.

Other alternative ways of silanization are reported to be carried out in the presence of solvents such as toluene, Xylene, ethylene dichloride, etc. Silica gel is suspended in toluene and appropriate amount of the silanating agent is added usually between 1:0.2 to 1:3 times (W/W) of silica gel and heated to 40-45° C. and then filtered and washed with methanol and water. Column chromatographic separation on silanized silica gel is applicable for purification of a number of compounds including 6-acetyl-TGS as well as TGS from reaction mixtures or from solutions done for any purpose.

### Example 1

### Preparation of TGS-6-Acetate

252.8 kg of PCl₅ was added slowly to 700 L of DMF taken in a Glass Lined Reactor with constant stirring at 20° C. temperature. The Vilsmeier reagent thus formed was then allowed to cool to 0° C. 80 kg of 75% pure sucrose-6-acetate dissolved in DMF was added slowly with constant stirring. The temperature during the addition was maintained between 0-5° C. In place of sucrose-6-acetate, an adaptation may include any other acylate or any other ester; and appropriate follow-up adaptations/modifications may have to be made accordingly.

After the addition of the sucrose-6-acetate, temperature was allowed to attain ambient temperature, usually around 30-35° C., and was stirred for 60 minutes. The reaction mass was then heated to 85° C., maintained for 60 minutes and further heated to 100° C. and maintained for 6 hrs. The reaction mass was again heated up to 115° C. and held for 90 minutes and then neutralized using 7% ammonia solution up to pH 6.5-7.0. The total volume was about 2000L of neutralized mass containing 1.4% of TGS-6-acetate

### Example 2

### Direct Purification of Neutralized Mass After Chlorination

The neutralized mass containing TGS-6-acetate from Example 1, was filtered in a filter press to remove all suspended matter and a clear filtrate was obtained. This reaction mixture is an aqueous composition with 20% DMF in it. 200 kg of silanized silica gel (hydrophobic) was slurried in phosphate buffer at pH 9.5 to 10.5 and was packed into a SS column (Here it is necessary to describe both the alternative procedures for silanizing silica gel) The silica gel was allowed to settle under the buffer without drying for 12 hours. Column Equilibration was started by passing about 600 L of buffer solution at pH 10.5 to 11.0. 250 L of filtered neutralized mass was loaded on the top of the silica gel bed packed in the SS column. Slight air pressure up to 0.5 to 0.8 kg/cm² was applied to push the neutralized mass through the silica gel bed. The flow rate from the column out put was adjusted to 200 LPH. As the neutralized mass passed completely through the top bed of the packed silica gel, the elution buffer. at pH 10.5 to 11.0 was added and the fractions were progressively eluted continuously.

Both equillibration and elution buffer are prepared by making a 0.1 molar concentration of sodium acetate solution and adjusting the pH of the solution to 10-11.5 using sodium hydroxide.

After the first 200 L of output from the column, the fractions started eluting out in pale brownish colour. 200 L fractions were collected separately and were analyzed for DMF and TGS-6-acetate content. The details of the fraction after HPLC and GC analysis are given in the table below. After the elution of TGS from the column, the passing of the elution buffer was stopped. Then 200 L of methanol was passed through the column to elute out all unwanted impurities and colour from the column. This was followed by passing 600 L of buffer at pH 10.5-11.0 for equilibration before loading the column with fresh neutralized mass to carry out the next cycle of purification.

The fractions from 4-8 as shown in the table was pooled together and were subjected to concentration by membrane filtration. The concentrated fraction up to 15% of TGS-6-acetate solution was taken for deacetylation using calcium hydroxide slurry. The deacetylation was monitored by TLC. After the deacetylation, the mass was extracted with 1:3 times of ethyl acetate. The organic layer containing TGS was charcoalized, concentrated and crystallized. The crystallized product was analyzed by HPLC. The purity was found to be 98.5% and the overall yield was found to be 25% of sucrose-6-ester input.

| | TGS-6-acetate | |
|---|---|---|
| | content | DMF content |
| 200 L Fractions | kg | Kg |
| Fraction 1 | 0.0 | 0.0 |
| Fraction 2 | 0.0 | 0.2 |
| Fraction 3 | 0.2 | 20.0 |
| Fraction 4 | 0.3 | 60.0 |
| Fraction 5 | 1.0 | 3.0 |
| Fraction 6 | 1.5 | 0.2 |
| Fraction 7 | 0.5 | 0.05 |
| Fraction 8 | 0.05 | 0.0 |

The other fractions containing DMF were taken for DMF recovery.

### Example 3

### Direct Purification of Neutralized Mass After Chlorination

The neutralized mass containing TGS-6-acetate from Example 1, was filtered in a filter press to remove all suspended matter and a clear filtrate was obtained. This filtrate was treated with 150 L of calcium hydroxide slurry in water at pH 9.0. The deacetylation was monitored by TLC. After the completion of deacetylation, the pH was adjusted to neutral and taken for purification by silanized silica gel chromatography.

250 L of the deacetylated mass was then taken for purification by hydrophobic silica gel chromatography. 200 kg of silanized silica gel (hydrophobic) was slurried in phosphate buffer at pH 9.5 to 10.5 and was packed into a SS column. The silica gel was allowed to settle under the buffer without drying for 12 hours. Column Equilibration was started by passing about 600 L of buffer solution at pH 10.5 to 11.0. 250 L of filtered deacetylated mass was loaded on the top of the silica gel bed packed in the SS column. Slight air pressure up to 0.5 to 0.8 kg/cm² was applied to push the neutralized mass through the silica gel bed. The flow rate from the column out put was adjusted to 200 LPH. As the deacetylated mass, which is an aqueous composition ordinarily containing 12-20% DMF passed completely through the top bed of the packed silica gel, the elution buffer at pH 10.5 to 11.0 was added and the fractions were progressively eluted continuously.

Both equillibration and elution buffer are prepared by making a 0.1 molar concentration of sodium acetate solution and adjusting the pH of the solution to 10-11.5 using sodium hydroxide. Fractions were collected as in the case of example 2 and results are as shown in the table below.

| | TGS content | DMF content |
|---|---|---|
| 200 L Fractions | kg | kg |
| Fraction 1 | 0.0 | 0.0 |
| Fraction 2 | 0.0 | 0.2 |
| Fraction 3 | 0.1 | 70.0 |
| Fraction 4 | 0.8 | 10.0 |
| Fraction 5 | 2.0 | 1.0 |
| Fraction 6 | 0.5 | 0.2 |
| Fraction 7 | 0.1 | 0.05 |

Fraction 5,6,7 were pooled together and was taken for concentration by membrane filtration. After concentration up to 15% of TGS content, the solution was extracted into 1:3 times of ethyl acetate. The organic extract was concentrated and crystallized. The overall yield obtained based on sucrose-6-acetate input was found to be 18%

The other fractions containing DMF were taken for DMF recovery.

### Example 4

### Purification After Extraction of Neutralized Mass

After the chlorination as stated in example 1, the neutralized mass thus obtained is directly extracted into 1:3.5 times of ethyl acetate and the layers were separated. The organic layer was separated out and was then subjected to concentration. The DMF partitioning into the ethyl acetate layer was about 10-12% of the total DMF content present in the neutralized mass.

The inorganic salts and rest of the DMF along with water was separated and taken for DMF recovery.

The syrup obtained after concentration of ethyl acetate was taken for purification by hydrophobic silica gel chromatography. The syrup was aqueous with about 28% as DMF. The loading of silanized silica gel and equilibration was followed as per example 2 and 3.

75 L of the syrup was loaded to the column and fractions were collected as per the earlier experiments. The pure fractions obtained were pooled, concentrated, deacetylated, extracted into ethyl acetate, charcoalized, concentrated and crystallized. The yield by this route was found to be 16.9%

### Example 5

Concentration and Extraction of Neutralized Mass

After the chlorination as stated in example 1 the neutralized mass thus obtained was concentrated to remove water and DMF under vacuum till the mass became syrupy. A large amount of inorganic salts also precipitated during the process. This was a semi solid paste, which was extracted into 1:3 times of ethyl acetate, and the solids were separated by filtration. The organic filtrate was then subjected to concentration. The DMF partitioning into the ethyl acetate layer was about 15% of the total DMF content present in the neutralized mass.

The syrup obtained after concentration of ethyl acetate was taken for purification by hydrophobic silica gel chromatography. The syrup was aqueous with about 30% DMF The loading of silanized silica gel and equilibration was followed as per example 2 and 3.

75 L of the syrup was loaded to the column containing 200 kg of silanized silica gel. and fractions were collected as per the earlier experiments. The pure fractions obtained were pooled, concentrated, deacetylated, extracted into ethyl acetate, charcoalized, concentrated and crystallized. The yield by this route was found to be 20.6%

### Example 6

### Purification of Neutralized Mass by Resin Chromatography

The neutralized mass containing TGS-6-acetate from Example 1, was filtered in a filter press to remove all suspended matter and a clear filtrate was obtained

200 kg of ADS 600 resin was slurried in phosphate buffer at pH 9.5 to 10.5 and was packed into a SS column. The resin was allowed to settle under the buffer without drying for 12 hours. Column Equilibration was started by passing about 600 L of buffer solution at pH 10.5 to 11.0. 250 L of filtered neutralized mass was loaded on the top of the resin packed in the SS column. Slight air pressure up to 0.5 to 0.8 kg/cm² was applied to push the neutralized mass through the resin bed. The flow rate from the column out put was adjusted to 200 LPH. As the neutralized mass passed completely through the top bed of the packed resin, the elution buffer.

at pH 10.5 to 11.0 was added and the fractions were progressively eluted continuously.

Both equillibration and elution buffer are prepared by making a 0.1 molar concentration of sodium acetate solution and adjusting the pH of the solution to 10-11.5 using sodium hydroxide.

After the first 160 L of output from the column, the fractions started eluting out in pale brownish colour. 150 L fractions were collected separately and were analyzed for DMF and TGS-6-acetate content. The details of the fraction after HPLC and GC analysis are given in the table below. After the elution of TGS from the column, the passing of the elution buffer was stopped. This was followed by passing 650 L of buffer at pH 10.5-11.0 for equilibration before loading the column with fresh neutralized mass to carry out the next cycle of purification.

The fractions from 4-8 as shown in the table was pooled together and were subjected to concentration by membrane filtration. The concentrated fraction up to 15% of TGS-6-acetate solution was taken for deacetylation using calcium hydroxide slurry. The deacetylation was monitored by TLC. After the deacetylation, the mass was extracted with 1:3 times of ethyl acetate. The organic layer containing TGS was charcoalized, concentrated and crystallized. The crystallized product was analyzed by HPLC. The purity was found to be 96.8% and the overall yield was found to be 28% of sucrose-6-ester input.

| | TGS-6-acetate | |
|---|---|---|
| | content | DMF content |
| 200 L Fractions | kg | Kg |
| Fraction 1 | 0.0 | 0.0 |
| Fraction 2 | 0.0 | 0.6 |
| Fraction 3 | 0.2 | 22.0 |
| Fraction 4 | 0.3 | 72.0 |
| Fraction 5 | 1.0 | 0.0 |
| Fraction 6 | 1.5 | 0.0 |
| Fraction 7 | 0.5 | 0.0 |
| Fraction 8 | 0.05 | 0.0 |

The other fractions containing DMF were taken for DMF recovery.

### Example 7

Recovery of DMF using silanated silica gel from waster water streams 3000 L of waste water stream containing 3%DMF and various inorganic salts were subjected to Falling Film evaporator to concentrate the stream up to a DMF content of 30%.

This concentrated solution was then loaded on to 300 kg of silanated silica gel packed in SS column. This silica gel was equilibrated with 600 L of acetate buffer at pH 10.5 to 11.0. After the loading of the DMF concentrate, the same acetate buffer was used for equilibration.

After the collection of the first void volume, DMF enriched fraction was collected in the first 120 L where about 70% solution was eluted. This solution was containing pure DMF and a colourless solution.

The subsequent fractions yielded other colour impurities and salts which were washed off the column.

The recovered DMF was then taken and used as such as 70% solution in water or was subjected to water removal by triple effect evaporator and purity of DMF recovered was found to be 98.7%. Same effect could also have been obtained by an alternative method involving use of distillation at reduced temperature, including but not limited to use of Falling Film Evaporators, Rising Film Evaporator and the like.

### Example 8

### Separation of 5,6-methylenedioxy-1-tetralone from DMF by Silanated silica gel chromatography

The mixture containing 5,6-methylenedioxy-1-tetralone in 20% DMF solution was subjected to separation by silanated silica gel chromatography.

500 ml of the sample was loaded on to 2.0 kg of silanated silica gel packed in a glass column. The silanated silica gel was equilibrated with acetate buffer at pH 10.5 to 11.0. After the loading of the sample, the mobile phase used was again acetate buffer at pH 10.5 to 11.0. 1000 ml of the buffer was passed through the column and the DMF in the sample was completely eluted out.

The DMF thus separated was subjected to water removal as mentioned in Example 7. After the water removal, the DMF purity was found to be 98%

The mobile phase was then changed to 40% of acetone in water and the 5,6-methylenedioxy-1-tetralone was eluted out, concentrated, extracted and crystallized.

1. A process of removal of a tertiary amide comprising preferably a N,N-dimethylformamide (DMF) from an aqueous composition by chromatography wherein;
a. the said aqueous composition is loaded on a fixed bed adsorbent which is non-polar, and
b. eluted by water at pH 7 to 12, preferably by an aqueous alkaline solution.

2. A process of claim 1 wherein the said aqueous composition contains, in addition to DMF, one or more of an organic molecule which is not an organic solvent including a chlorinated sucrose-6-ester, a chlorinated sucrose, and
a. the said non-polar fixed bed adsorbent is silanated silica gel;
b. the said aqueous alkaline solution is one of the following:
i. water, more preferably a buffer, adjusted to alkaline pH within range of 7 to12, more preferably of 9.5 to 11.5, still more preferably from 10.5 to 11.5;
ii. water with acetonitrile or eluted by acetone preferably in 5% concentration v/v;
iii. methanol in water preferably 2% concentration v/v, or
iv. an organic solvent miscible in water in any ratio.

3. A process of claim 2 comprising steps of:
a. Attaching a silane group to silica gel by using one or more of a process of silanization silica gel.
b. slurrying silanized silica gel in phosphate buffer at pH 9.5 to 10.5 and packing into a stainless steel column and settling for about 12 hours,
c. equilibrating column by a buffer of pH 10.5 to 11.0,
d. loading the DMF containing aqueous liquid composition at or around neutral pH on the top of the silica gel bed packed in the SS column,
e. applying slight air pressure, preferably up to 0.5 to 0.8 kg/cm² to push the neutralized mass through the silica gel bed
f. passing the said aqueous composition containing DMF completely through the top bed of the packed silica gel,
g. adding the elution buffer at around pH 10.5 to 11.0,
h. collecting separately DMF containing fractions
i. collecting other components of the loaded aqueous composition separately therafter.

4. A process of claim 3 wherein the said process of silanization of silica gel comprises one or more of a method including:
a. silanization by reacting with vapors of a silanating agent including:
i. by allowing the vapors of a silanating agent, preferably trimethylchlorosilane or dimethylchlorosilane to coat onto a silica gel in a closed environment for a period time, preferably of 48 hours,
ii. dispersing the treated silica gel in water and collecting the silanated silica gel which floats at the top of the solution
b. silanization by reacting with trimethylchlorosilane in presence of a solvent comprising steps of:
i. slurrying silica gel in an organic solvent, preferably toluene,
ii. adding to it trimethylchlorosilane and mixing thoroughly, preferably by stirring, at a slightly elevated temperature for a period of time, preferably at around 45° C. for about two hours,
iii. filtering off the silica gel cake and washing it with methanol thoroughly to remove traces of the said organic solvent used, followed by water wash,

5. A process of claim 1 wherein the said aqueous composition contains, in addition to DMF, one or more of an organic molecule which is not an organic solvent including a chlorinated sucrose-6-ester, a chlorinated sucrose, and
a. the said non-polar fixed bed adsorbent is a polyacrilyc based resin ADS600;
b. the said aqueous alkaline solution is one of the following:
i. water, more preferably a buffer, adjusted to alkaline pH within range of 7 to12, more preferably of 9.5 to 11.5, still more preferably from 10.5 to 11.5;
ii. water with acetonitrile or eluted by acetone preferably in 5% concentration v/v;
iii. methanol in water preferably 2% concentration v/v, or
iv. an organic solvent miscible in water in any ratio.

6. A process of claim 1, wherein the said chlorinated sucrose includes 4,1′,6′ trichlorogalactosucrose (TGS) and the said precursor or derivative of chlorinated sucrose includes 6-acetyl-4,1′,6′ trichlorogalactose (6-acetyl-TGS),

7. A process of claim 1 or claim 2 or claim 5 wherein the said aqueous composition subjected to chromatographic separation is derived from in one or more of following means:
a. dissolution of the TGS or 6-acetyl-TGS, in an aqueous medium.
b. as a process stream, an aqueous reaction mixture, derived from a process of production of TGS or 6-acetyl-TGS.

8. A process of claim 2 wherein the said chlorinated sucrose includes 4,1′,6′ trichlorogalactosucrose (TGS) and the said precursor or derivative of chlorinated sucrose includes 6-acetyl-4,1′,6′ trichlorogalactose (6-acetyl-TGS).

9. A process of claim 5 wherein the said chlorinated sucrose includes 4,1′,6′ trichlorogalactosucrose (TGS) and the said precursor or derivative of chlorinated sucrose includes 6-acetyl-4,1′,6′ trichlorogalactose (6-acetyl-TGS).

10. A process of claim 2 wherein the said aqueous composition subjected to chromatographic separation is derived from in one or more of following means:
a. dissolution of the TGS or 6-acetyl-TGS, in an aqueous medium.
b. as a process stream, an aqueous reaction mixture, derived from a process of production of TGS or 6-acetyl-TGS.

11. A process of claim 5 wherein the said aqueous composition subjected to chromatographic separation is derived from in one or more of following means:
a. dissolution of the TGS or 6-acetyl-TGS, in an aqueous medium.
b. as a process stream, an aqueous reaction mixture, derived from a process of production of TGS or 6-acetyl-TGS.
